(19) United States
(12) Patent Application Publication
(10) Pub. No.: US 2025/0043274 A1
(43) Pub. Date: Feb. 6, 2025

(54) Antibody Protein Product Expression Constructs for High Throughput Sequencing

(71) Applicant: AMGEN INC., Thousand Oaks, CA (US)

(72) Inventors: Sarah Van Driesche, Thousand Oaks, CA (US); Amy Pandya Jones, Thousand Oaks, CA (US)

(21) Appl. No.: 18/836,103

(22) PCT Filed: Feb. 8, 2023
(86) PCT No.: PCT/US23/12574

§ 371 Date: Aug. 6, 2024

Related U.S. Application Data
(60) Provisional application No. 63/308,922, filed on Feb. 10, 2022.

(51) Int. Cl. C12N 15/10

(52) U.S. Cl. CPC C12N 15/1065; C12N 15/1068

(57) ABSTRACT

The disclosure provides for a polynucleotide molecule encoding an antibody protein product, wherein the polynucleotide molecule comprises i) a nucleotide sequence specific for the addition of a molecular barcode by a template switching reverse transcriptase (RT) and a unique molecular identifier (UMI) barcode, and a nucleotide sequence specific for a universal RT primer to facilitate high throughput sequencing.

This application claims priority benefit to U.S. provisional application No. 63/308,922, filed Feb. 10, 2022, which is incorporated by reference herein in its entirety.

### FIELD

The disclosure provides for a polynucleotide molecule encoding an antibody protein product, wherein the polynucleotide molecule comprises a nucleotide sequence specific for the addition of a molecular barcode by a template switching reverse transcriptase (RT), and a unique molecular identifier (UMI) barcode, and a nucleotide sequence specific for a universal RT primer to facilitate high throughput sequencing.

### BACKGROUND

During the development of therapeutic antibodies, much time and resources are invested in the identification of potentially problematic physicochemical properties of antibodies to mitigate the risk for costly late-stage failures in the clinic. High throughput sequencing, also known as next-generation sequencing, is one method of early evaluation of antibody protein product expressing clones. For example, high throughput sequencing may be used to evaluate the diversity of antibodies encoded in a combination library. In addition, high throughput screening allows for selection of antibody protein products allow for identification of sequence encoding proteins having the desired characteristics, such as high-affinity antibodies.

Fluidic systems may be used for carrying out high throughput sequencing of antibody protein products. The use of optical barcodes and an optofluidic systems provide for high throughput single cell screening capability based on nanofluidic and opto-electronic positioning technology. This technology is based on light-induced electrokinetics that gives rise to designated forces on both solid and fluidic structures (Jorgolli et al., Biotechnol Bioeng 2019, 116 (9), 2393-2411). For example, commercially available fluidic devices, such as the integrated technology of the Berkeley Lights (BLI) Beacon® Optofluidic System (Emeryville, CA) have the flexibility and capability for a broad array of applications applicable to commercial large molecule drug development, including antibody discovery, clonal selection, gene editing, linking phenotype to genotype, and cell line development.

The use of an optical fluidic system enables high throughput linkage or phenotype to genotype with rapid CEPA cycling of large panels (10,000's of molecules) and machine learning. As antibody protein products are large complex molecules, there are difficulties in effectively carrying out high throughput sequencing of expression constructs. For example, a conventional optical barcode typically allows pooled export of up to 12 beads at a time for sequencing off chip. Due to the size of the construct expressing the antibody protein product, using standard constructs and sequence methods, only the ends of the polynucleotide molecule are sequenced and the entire polynucleotide molecule can only be sequenced if exported one bead at a time rather than in pools. Sequencing off chip would thus be limited to a few thousand sequences if pooled, a few hundred if not pooled. Thus, there is a need for developing antibody protein product expression construct that can effectively be sequenced using high throughput sequencing techniques or next generation sequencing techniques.

### SUMMARY

The disclosure provides for polynucleotide expression constructs for high throughput screening of clones during antibody protein product development. The disclosed polynucleotide sequences include a universal molecular identification (UMI) barcode and a molecular barcode such as an optical barcode during cloning. By reading both the molecular (e.g., optical) barcode and the UMI barcode together, the identity of the molecule sequence can be determined using an optical fluidic device, for example the barcode can be linked to the particular sequestration pen or fluidic device chamber.

The disclosure provides for a polynucleotide molecule encoding an antibody protein product, wherein the polynucleotide molecule comprises i) a nucleotide sequence specific for the addition of a molecular barcode by a template switching reverse transcriptase (RT), ii) a unique molecular identifier (UMI) barcode that is different than the molecular barcode, iii) a nucleotide sequence encoding a light chain polypeptide of the antibody protein product, iv) a nucleotide sequence encoding a heavy chain polypeptide of the antibody protein product, and v) a nucleotide sequence specific for a universal RT primer.

A template switching reverse transcriptase (RT) refers to a RT that adds a few non-templated nucleotides after it reaches the 5′ end of the RNA template (e.g., 5′CCC).

These non-templated nucleotides can anneal to a template switching oligo (TSO) with a known sequence, prompting the reverse transcriptase to switch template from RNA to the TSO. For example, a TSO may comprise 3 riboguanosines at its 3′ end, which may anneal to a 5′ CCC sequence of the RNA template. The resulting cDNA contains a known sequence (complementary to the sequence of the TSO) attached to the cDNA's 3′ end. In the disclosed polynucleotide molecules, the “nucleotide sequence specific for the addition of a molecule barcode by a template switching RT” refers to a nucleotide sequence that can anneal to a TSO, wherein the TSO comprises a molecular barcode.

A “molecular barcode” refers to a unique sequence of nucleotide that is a unique identifier for the polynucleotide sequence. For example, molecular barcodes are a string of random nucleotides, or partially degenerate nucleotides, or defined nucleotides that may be used to identify a target molecule during DNA processing and/or sequencing. A molecular barcode is useful for quantitative sequencing applications and also for genomic variant detection. Molecular barcode information in conjunction with alignment coordinates enables grouping of sequencing data into read families representing individual sample DNA or RNA fragments.

The term “unique molecular identifier” refers to a molecular barcode that is used to identify the antibody protein product producing clone. In addition, the molecular barcode inserted by the template switching RT refers to a nucleotide sequence that provides an additional level of identification, such as a nucleotide sequence that identifies the pen, well or chamber from which the antibody protein product producing clone originated.

The “universal RT primer” is a primer having a sequence complementary to nucleotide sequences that are very common in a particular set of DNA molecules and cloning vectors. Thus, the universal RT primer may be used to reverse transcribe a collection of different DNA molecules that all share the common nucleotide sequence for the universal RT primer. These primers are able to bind to a wide variety of DNA templates. A “nucleotide sequence specific for a universal RT primer” is a nucleotide sequence that anneals to a universal RT primer.

In any of the polynucleotide molecules, the molecular barcode, such as the UMI or the optical barcode, comprises a random 6mer, 7mer, 8mer, 9mer, 10mer, 11mer, 12mer, 13mer, 14mer or 15mer, including ranges between any two of the listed values, for example a 6mer-16mer, 10mer-16mer, or 12mer-16mer. In some embodiments, the optical barcode comprises a random nucleic acid sequence that is at least a 6mer, 7mer, 8mer, 9mer, 10mer, 11mer, 12mer, 13mer, 14mer or 15mer in length.

In any of the polynucleotide molecules described herein, the polynucleotide molecule further comprises a promoter sequence.

In addition, in any of the polynucleotide molecules described herein, the polynucleotide molecule further comprises at least two internal ribosome entry site (IRES) sequences. For example, an IRES may be disposed between two different open reading frames, such as those of a heavy chain polypeptide and light chain polypeptide, or between those of a polypeptide of the antibody protein product and a selection gene product.

In addition, in any of the polynucleotide molecules described herein, the polynucleotide molecule further comprises at least IRES sequence and at least one promoter. The disclosure also provides any of the polynucleotide molecules further comprising at least two promoters.

In addition, in any of the polynucleotide molecules described herein, the polynucleotide molecule further comprises a nucleotide encoding a selection gene product, such as puromycin-N-acetyltransferase.

In an exemplary polynucleotide molecule described herein, the nucleotide sequence specific for the universal RT primer is disposed between the nucleotide sequence encoding the light chain polypeptide and the nucleotide sequence encoding the heavy chain polypeptide.

In another exemplary polynucleotide molecule described herein, the nucleotide sequence specific for the universal RT primer is downstream of the nucleotide sequence encoding the light chain polypeptide of the antibody protein product.

In some embodiments, in any of the disclosed polynucleotide molecules, the nucleotide sequence specific for the addition of a molecular barcode by a template switching reverse transcriptase is adjacent to the UMI barcode. The term “adjacent” refers to an element that is disposed immediately downstream or immediately upstream of the reference element. For example, in any of the disclosed polynucleotide molecules, the nucleotide sequence specific for the addition of a molecular barcode by a template switching reverse transcriptase may be immediately upstream of the UMI barcode.

In some embodiments, in any of the disclosed polynucleotide molecules, the UMI barcode is immediately upstream from the nucleotide sequence encoding the light chain polypeptide of the antibody protein product.

The disclosure provides for polynucleotide molecule in which the nucleotide sequence specific for the RT universal primer is immediately downstream of the nucleotide sequence encoding the light chain polypeptide of the antibody protein product.

In any of the disclosed polynucleotide molecules, the polynucleotide comprises an IRES immediately downstream of the sequence specific for the RT universal primer.

In any of the disclosed polynucleotide molecules, the polynucleotide comprises an IRES immediately downstream of the nucleotide sequence encoding the heavy chain polypeptide of the antibody protein product.

In any of the disclosed polynucleotide molecules, the heavy chain polypeptide comprises a heavy chain variable region; and the light chain polypeptide comprises a light chain variable region.

In any of the disclosed polynucleotide molecules, (a) the nucleotide sequence encoding the light chain polypeptide of the antibody protein product encodes: a light chain variable region, and a light chain constant region immediately downstream of the light chain constant region; and (b) the nucleotide sequence encoding the heavy chain polypeptide of the antibody protein product encodes: a heavy chain variable region, and a heavy chain constant region immediately downstream of the heavy chain constant region.

In some of the disclosed polynucleotide molecules, the nucleotide sequence encoding the light chain polypeptide of the antibody protein product is upstream of the nucleotide sequence encoding the heavy chain polypeptide of the antibody protein product.

In some of the disclosed polynucleotide molecules, the nucleotide sequence encoding the light chain polypeptide of the antibody protein product is downstream of the nucleotide sequence encoding the heavy chain polypeptide of the antibody protein product.

The disclosure provides for a polynucleotide molecule encoding an antibody protein product, wherein the polynucleotide molecule comprises in the 5′ to 3′ direction: i) a promoter, ii) a nucleotide sequence specific for the addition of a molecular barcode by a template switching reverse transcriptase, iii) a unique molecular identifier (UMI) barcode that is different from the molecular barcode, iv) a nucleotide sequence encoding a the first polypeptide of an antibody protein product, v) a nucleotide sequence specific for a reverse transcriptase (RT) universal primer, vi) a first IRES, vii) a nucleotide sequence encoding a second polypeptide of the antibody protein product, viii) a nucleotide sequence encoding the constant domain of the heavy chain of the antibody protein product, ix) a second IRES and x) a nucleotide sequence encoding a selection gene product, such as puromycin-N-acetyltransferase.

In addition, the disclosure provides for a polynucleotide molecule encoding an antibody protein product, wherein the polynucleotide sequence comprises, i) a sequencing primer annealing site, ii) a unique molecular identifier barcode, iii) a nucleotide sequence encoding a light chain polypeptide of the antibody protein product, iv) a nucleotide sequence encoding a heavy chain polypeptide of the antibody protein product; and iv) a nucleotide sequence specific for a universal reverse transcriptase (RT) primer.

In any of the disclosed polynucleotide molecules, the molecular barcode is an optical barcode. An optical barcode refers to a molecular barcode that is visually detectable. In some embodiments, the optical barcode (i) is identifiable by determining its polynucleotide sequence, and (ii) is identifiable by annealing to a polynucleotide probe comprising one or more optical moieties. The optical moiety may be a fluroprobe, fluorescent probe or a colormetric probe. Exemplary optical barcodes are provided in FIGS. 4 and 5. As such, polynucleotide molecules comprising an optical barcode may be annealed to a polynucleotide probe comprising one or more optical moieties, and the identifying information for the molecular barcode may be identified in situ, for example by light absorption or emission.

In any of the disclosed polynucleotide molecules, the nucleotide sequence specific for the addition of an optical barcode is configured to receive the optical barcode conjugated to a solid support.

In addition, in any of the disclosed polynucleotide molecules, the nucleic acid specific for the addition of a molecular barcode by a template switching reverse transcriptase is configured for the addition of the optical barcode from by a template switching oligonucleotide from a template optical barcode conjugated to a solid support.

In any of the disclosed polynucleotide molecules, the 5′ end of the nucleotide sequence specific the for the addition of a molecular barcode comprises the polynucleotide sequence CCC. In any of the disclosed polynucleotide molecules, the 5′ end of the nucleotide sequence specific the for the addition of a molecular barcode comprises a polynucleotide sequence that is a reverse complement to a TSO.

In any of the disclosed polynucleotide molecules, the molecular barcode is configured for reverse transcription by template switching reverse transcriptase primed by the RT universal primer.

In some embodiments, any of the disclosed polynucleotide molecules, the nucleotide sequence specific for a reverse transcriptase (RT) universal primer is configured to anneal to free universal primer that is not disposed on the solid support, and wherein the annealed universal primer is configured for reverse transcription of the nucleotide barcode by the reverse transcriptase.

In various embodiments, the disclosed polynucleotide molecules wherein the solid support is a bead or a microsphere, a membrane, nanofiber, nanotube, resin or agarose. For example, the solid support may be polymer-based e.g. polystyrene beads, poly(lactide-co-glycolide) (PLGA) beads, polyethylene oxide (PEO) beads, polyethylene glycol (PEG) beads, polyvinyl alcohol (PVA) beads, or may be metal-based e.g. gold beads. In addition, the solid support may be chitosan, dextran, alginate, gadolinium-based, carbon-based, silica-based or iron-based. When the solid support is a resin, the resin may be a polymeric resin such as cellulose, polystyrene, agarose, polyacrylamide or agarose.

In any of the disclosed polynucleotide molecules (a) the first polypeptide of the antibody protein product comprises a light chain polypeptide and the second polypeptide of the antibody protein product comprises a heavy chain polypeptide; or (b) the first polypeptide of the antibody protein product comprises a heavy chain polypeptide and the second polypeptide of the antibody protein product comprises a light chain polypeptide.

For example, in some disclosed polynucleotide molecules, the light chain polypeptide comprises a light chain variable region, and the heavy chain polypeptide comprises a heavy chain variable region.

In addition, in some of the disclosed polynucleotide molecules, the nucleotide sequence encoding the light chain polypeptide of the antibody protein product encodes: a light chain variable region, and a light chain constant region immediately downstream of the light chain constant region; and the nucleotide sequence encoding the heavy chain polypeptide of the antibody protein product encodes: a heavy chain variable region, and a heavy chain constant region immediately downstream of the heavy chain constant region.

In any of the disclosed polynucleotide molecules encoding an antibody protein product, the antibody protein product comprises or consists of a large peptide, antibody, antibody fragment, antibody fusion peptide or antigen-binding fragment thereof. For example, the antibody is a polyclonal or monoclonal antibody.

The disclosure also provides for method of screening clones expressing an antibody protein product comprising pooling clones comprising any of the polynucleotide molecules disclosed herein, polymerizing a DNA of the pooled clones and sequencing the DNA in a fluidic device. For example, the polynucleotide molecule may comprise RNA. For example, polymerizing the DNA of the pooled clones comprises reverse transcribing the polynucleotide molecule with a template switching reverse transcriptase.

The disclosure also provides for methods comprising annealing any of the polynucleotide molecules disclosed herein to template comprising optical barcode, such as wherein said template is conjugated to a solid support; annealing a universal reverse transcriptase primer to the polynucleotide molecule; and extending the annealed universal reverse transcriptase primer with a template switching reverse transcriptase, thereby producing a cDNA of the polynucleotide molecule comprising the UMI barcode and the optical barcode. For example, this method is performed in a fluidic device, optionally the fluidic device comprises or consists of a microfluidic chip or sequestration pen.

Also provided are any of the disclosed methods, further comprising detecting the presence of a molecular barcode (such as an optical barcode) and/or a UMI barcode.

In any of the disclosed methods, the method can be carried out with any fluidic system, fluidic device or fluidic apparatus known in the art. For example, the method may be carried out in situ in the fluidic system, fluidic device or fluidic apparatus. A fluidic device (or fluidic apparatus) is a device that includes one or more discrete circuits configured to hold a fluid, each circuit comprised of fluidically interconnected circuit elements. The circuit element including but not limited to region(s), flow path(s), channel(s), chamber(s), and/or pen(s), and at least one port configured to allow the fluid to flow into and/or out of the fluidic device. The fluidic circuit may be configured to have a first end fluidically connected with a first port (e.g., an inlet) in the fluidic device and a second end fluidically connected with a second port (e.g., an outlet) in the fluidic device or connected to a second fluidic device or a second region, flow path, channel, chamber or pen in the fluidic device. The fluidic device may be a microfluidic device, through other scales such as nano-scale may also be suitable. For example, the fluidic device may be a microfluidic chip, microfluidic channel, microfluidic cell, nanofluidic chip, nanofluidic channel, nanofluidic cell or sequestration pen. In some embodiments, the fluidic system comprises a multi-well plate, for example a 96- or 384-will plate. The multi-well plate may be in fluid communication with a circuit.

For a microfluidic device, the circuit will include a flow region, which may include a microfluidic channel, and at least one chamber, and will hold a volume of fluid of less than about 1 mL, e.g., less than about 750, 500, 250, 200, 150, 100, 75, 50, 25, 20, 15, 10, 9, 8, 7, 6, 5, 4, 3, or 2 μL. In certain embodiments, the circuit holds about 1-2, 1-3, 1-4, 1-5, 2-5, 2-8, 2-10, 2-12, 2-15, 2-20, 5-20, 5-30, 5-40, 5-50, 10-50, 10-75, 10-100, 20-100, 20-150, 20-200, 50-200, 50-250, or 50-300 μL. The circuit may be configured to have a first end fluidically connected with a first port (e.g., an inlet) in the microfluidic device and a second end fluidically connected with a second port (e.g., an outlet) in the microfluidic device.

As used herein, a “nanofluidic device” or “nanofluidic apparatus” is a type of fluidic device having a fluidic circuit that contains at least one circuit element configured to hold a volume of fluid of less than about 1 μL, e.g., less than about 750, 500, 250, 200, 150, 100, 75, 50, 25, 20, 15, 10, 9, 8, 7, 6, 5, 4, 3, 2, 1 nL or less. A nanofluidic device may comprise a plurality of circuit elements (e.g., at least 2, 3, 4, 5, 6, 7, 8, 9, 10, 15, 20, 25, 50, 75, 100, 150, 200, 250, 300, 400, 500, 600, 700, 800, 900, 1000, 1500, 2000, 2500, 3000, 3500, 4000, 4500, 5000, 6000, 7000, 8000, 9000, 10,000, or more). In certain embodiments, one or more (e.g., all) of the at least one circuit elements is configured to hold a volume of fluid of about 100 μL to 1 nL, 100 μL to 2 nL, 100 μL to 5 nL, 250 μL to 2 nL, 250 μL to 5 nL, 250 μL to 10 nL, 500 μL to 5 nL, 500 μL to 10 nL, 500 μL to 15 nL, 750 μL to 10 nL, 750 μL to 15 nL, 750 μL to 20 nL, 1 to 10 nL, 1 to 15 nL, 1 to 20 nL, 1 to 25 nL, or 1 to 50 nL. In other embodiments, one or more (e.g., all) of the at least one circuit elements is configured to hold a volume of fluid of about 20 nL to 200 nL, 100 to 200 nL, 100 to 300 nL, 100 to 400 nL, 100 to 500 nL, 200 to 300 nL, 200 to 400 nL, 200 to 500 nL, 200 to 600 nL, 200 to 700 nL, 250 to 400 nL, 250 to 500 nL, 250 to 600 nL, or 250 to 750 nL.

A “fluidic channel” or “flow channel” as used herein refers to a flow region of a fluidic device having a length that is significantly longer than both the horizontal and vertical dimensions. For example, the flow channel can be at least 5 times the length of either the horizontal or vertical dimension, e.g., at least 10 times the length, at least 25 times the length, at least 100 times the length, at least 200 times the length, at least 500 times the length, at least 1,000 times the length, at least 5,000 times the length, or longer. In some embodiments, the length of a flow channel is in the range of from about 50,000 microns to about 500,000 microns, including any range there between. In some embodiments, the horizontal dimension is in the range of from about 100 microns to about 1000 microns (e.g., about 150 to about 500 microns) and the vertical dimension is in the range of from about 25 microns to about 200 microns, e.g., from about 40 to about 150 microns. It is noted that a flow channel may have a variety of different spatial configurations in a fluidic device, and thus is not restricted to a perfectly linear element. For example, a flow channel may include one or more sections having any of the following configurations: curve, bend, spiral, incline, decline, fork (e.g., multiple different flow paths), and any combination thereof. In addition, a flow channel may have different cross-sectional areas along its path, widening and constricting to provide a desired fluid flow therein.

### BRIEF DESCRIPTION OF THE DRAWINGS

FIG. 1 provides a schematic of a polynucleotide molecule for 5′ sequencing.

FIGS. 2A-2B provide schematics of polynucleotide molecules designed for on-chip sequencing.

FIGS. 3A-3B provide schematics of polynucleotide molecules designed for 3′ sequencing.

FIG. 4 provides exemplary 5′ optical barcode chemistry.

FIG. 5 provides exemplary 3′ optical barcode chemistry.

FIG. 6 provide a schematic of conventional polynucleotide molecules that are subject to limitations if attempts are made to sequence using 3′ sequencing kits.

FIG. 7 provide a schematic of conventional polynucleotide molecules that are subject to limitations if attempts are made to sequence using 5′ sequencing kits.

### DETAILED DESCRIPTION

The disclosure provides for a polynucleotide molecule encoding an antibody protein product, wherein the polynucleotide molecule comprises i) a nucleotide sequence specific for the addition of a molecular barcode by a template switching reverse transcriptase (RT) and a unique molecular identifier (UMI) barcode (that is different from the molecular barcode), and a nucleotide sequence specific for a universal RT primer to facilitate high throughput sequencing.

The disclosed polynucleotide sequences have an advantage of functioning with commercially available constructs. Conventional template switching reverse transcriptase limits reverse transcription to a short number of nucleotides e.g. about 500 to about 1000 nucleotides. The polynucleotide molecules disclosed herein position a universal RT primer downstream of polynucleotide sequence encoding a polypeptide of an antibody protein product (such as the constant domain of a light chain). This allows for flowing custom primers into a fluidic device, and therefore the primer does not need to be on a bead or solid support. Beads comprising oligodT may be used to capture the mRNA of the polynucleotide sequence. The custom primer will then bind to the captured mRNA and initiate extension closer to the 5′ end of the polynucleotide molecule.

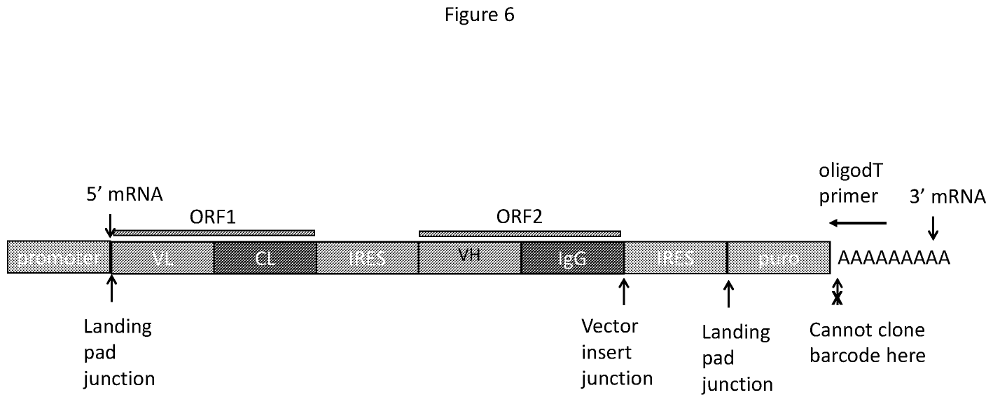

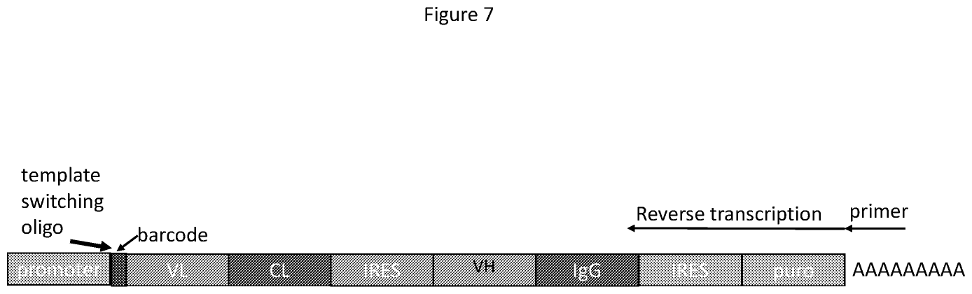

For example, commercially available 3′ sequencing kits do not effectively sequence the commercially available landing pad constructs having a polyadenylation signal (pA) in the cell host landing pad, far downstream of the cloning insert junction. A UMI barcode inserted at the insert junction is not close enough to the optical barcode, which would be part of a dT oligo, downstream of the pA (see, e.g. FIG. 6). In addition, commercially available 5′ sequencing kits do not effectively sequence the commercially available landing pad constructs because the primer is too far from the molecular barcode (see, e.g. FIG. 7). There is about 50% decrease in sequencing depth of the polynucleotide encoding the variable domain of the heavy chain of an antibody protein product compared to the sequencing depth of the polynucleotide sequence encoding the variable domain of the light chain of an antibody protein product. A more significant drop off in sequencing depth is expect if the reverse transcriptase was trying to transcribe a 4 kB insert with a IRES or promoter sequence in the middle of the polynucleotide molecule.

Fluidic devices may allow for growing and expanding a single cell within a chamber or sequestration pen, which in turn allow for clonal selection of the cell producing the antibody protein product to be sequenced. The clonal selection allows for selection of the clones for large-scale protein production and purification during drug discovery and biologic drug manufacturing, e.g. antibody production. The disclosed methods also allow for continual analysis of the cells as they are expanding and the assays can be repeated on the same growing cell.

A colony of biological cells is “clonal” if all of the living cells in the colony that are capable of reproducing are daughter cells derived from a single progenitor cell. In certain embodiments, all the daughter cells in a clonal colony are derived from the single progenitor cell by no more than 10 divisions. In other embodiments, all the daughter cells in a clonal colony are derived from the single progenitor cell by no more than 14 divisions. In other embodiments, all the daughter cells in a clonal colony are derived from the single progenitor cell by no more than 17 divisions. In other embodiments, all the daughter cells in a clonal colony are derived from the single progenitor cell by no more than 20 divisions. The term “clonal cells” refers to cells of the same clonal colony.

As used herein, a “colony” of biological cells refers to 2 or more cells (e.g. about 2 to about 20, about 4 to about 40, about 6 to about 60, about 8 to about 80, about 10 to about 100, about 20 about 200, about 40 about 400, about 60 about 600, about 80 about 800, about 100 about 1000, or greater than 1000 cells).

As used herein, the term “maintaining (a) cell(s)” refers to providing an environment comprising both fluidic and gaseous components and, optionally a surface, that provides the conditions necessary to keep the cells viable and/or expanding.

As used herein, the term “expanding” when referring to cells, refers to increasing in cell number.

### Antibody Protein Product

The disclosure provides for polynucleotide molecules encoding an antibody protein product. Antibody protein products include an antibody, bispecific T-cell engager (BiTE®) molecule, antibody fragment, antibody fusion peptide or antigen-binding fragment thereof, or peptide. In related embodiments, the antibody is a polyclonal or monoclonal antibody. As used herein, the term “antibody protein product” refers to antibodies, as well as any one of several antibody alternatives which in various instances is based on the architecture of an antibody but is not found in nature.

An “antibody” is a subgenus of antibody protein product. It refers to refers to an immunoglobulin of any isotype with specific binding to the target antigen, and includes, for instance, monoclonal antibodies. Antibodies may be of any suitable host species, for example, chimeric, humanized, fully human, fully mouse, fully rabbit, or fully llama. An antibody generally comprises two full-length heavy chains and two full-length light chains. For example, human antibodies can be of any isotype, including IgG (including IgG1, IgG2, IgG3 and IgG4 subtypes), IgA (including IgA1 and IgA2 subtypes), IgM and IgE. In some aspects, the antibody protein product has a molecular-weight within the range of at least about 12 kDa-10 MDa, for example at least about 12 kDa-5 MDa, 12 kDa-1 MDa, 12 kDa-750 KDa, at least about 12 kDa-250 kDa, or at least about 12 kDa-150 kDa. In certain aspects, the antibody protein product has a valency (n) range from monomeric (n=1), to dimeric (n=2), to trimeric (n=3), to tetrameric (n=4), if not higher order valency. Antibody protein products in some aspects are those based on the full antibody structure and/or those that mimic antibody fragments which retain full antigen-binding capacity, e.g., scFvs, Fabs and VHH/VH (discussed below). The smallest antigen binding antibody fragment that retains its complete antigen binding site is the Fv fragment, which consists entirely of variable (V) regions. A soluble, flexible amino acid peptide linker is used to connect the V regions to a scFv (single chain fragment variable) fragment for stabilization of the molecule, or the constant (C) domains are added to the V regions to generate a Fab fragment [fragment, antigen-binding]. Both scFv and Fab fragments can be easily produced in host cells, e.g., prokaryotic host cells. Other antibody protein products include disulfide-bond stabilized scFv (ds-scFv), single chain Fab (scFab), as well as di- and multimeric antibody formats like dia-, tria- and tetra-bodies, or minibodies (miniAbs) that comprise different formats comprising scFvs linked to oligomerization domains. The smallest fragments are VHH/VH of camelid heavy chain Abs as well as single domain Abs (sdAb) including UniDab® construct-containing molecules and UniAb® constructs (TeneoBio). The building block that is most frequently used to create novel antibody formats is the single-chain variable (V)-domain antibody fragment (scFv), which comprises V domains from the heavy and light chain (VH and VL domain) linked by a peptide linker of ˜15 amino acid residues. A peptibody or peptide-Fc fusion is yet another antibody protein product. The structure of a peptibody comprises a biologically active peptide grafted onto an Fc domain. Peptibodies are well-described in the art. See, e.g., Shimamoto et al., mAbs 4(5): 586-591 (2012). Other antibody protein products include a single chain antibody (SCA); a diabody; a triabody; a tetrabody; bispecific or trispecific antibodies, and the like. Bispecific antibodies can be divided into five major classes: BslgG, appended IgG, BsAb fragments, bispecific fusion proteins and BsAb conjugates. See, e.g., Spiess et al., Molecular Immunology 67(2) Part A: 97-106 (2015). In exemplary aspects, the antibody protein product comprises or consists of a bispecific T cell engager (BiTE®) molecule, which is an artificial bispecific monoclonal antibody. BiTE® molecules are fusion proteins comprising two scFvs of different antibodies. One binds to CD3 and the other binds to a target antigen. BiTE® molecules are known in the art. See, e.g., Huehls et al., Immuno Cell Biol 93(3): 290-296 (2015); Rossi et al., MAbs 6(2): 381-91 (2014); Ross et al., PLoS One 12(8): e0183390.

### Polynucleotide Molecules

The term “recombinant” indicates that the material (e.g., a nucleic acid or a polypeptide) has been artificially or synthetically (i.e., non-naturally) altered by human intervention. The alteration can be performed on the material within, or removed from, its natural environment or state. For example, a “recombinant nucleic acid” is one that is made by recombining nucleic acids, e.g., during cloning, DNA shuffling or other well known molecular biological procedures. Examples of such molecular biological procedures are found in Maniatis et al., Molecular Cloning. A Laboratory Manual. Cold Spring Harbor Laboratory, Cold Spring Harbor, N.Y (1982). A “recombinant DNA molecule,” is comprised of segments of DNA joined together by means of such molecular biological techniques. The term “recombinant protein” or “recombinant polypeptide” as used herein refers to a protein molecule which is expressed using a recombinant DNA molecule. A “recombinant host cell” is a cell that contains and/or expresses a recombinant nucleic acid.

The term “polynucleotide” or “nucleic acid” includes both single-stranded and double-stranded nucleotide polymers containing two or more nucleotide residues. The nucleotide residues comprising the polynucleotide can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phosphoraniladate and phosphoroamidate.

The term “oligonucleotide” means a polynucleotide comprising 200 or fewer nucleotide residues. In some embodiments, oligonucleotides are 10 to 60 bases in length. In other embodiments, oligonucleotides are 12, 13, 14, 15, 16, 17, 18, 19, or 20 to 40 nucleotides in length. Oligonucleotides may be single stranded or double stranded, e.g., for use in the construction of a mutant gene. Oligonucleotides may be sense or antisense oligonucleotides. An oligonucleotide can include a label, including an isotopic label (e.g., ¹²⁵I, ¹⁴C, ¹³C, ³⁵S, ³H, ²H, ¹³N, ¹⁵N, ¹⁸O, ¹⁷O, etc.), for ease of quantification or detection, a fluorescent label, a hapten or an antigenic label, for detection assays. Oligonucleotides may be used, for example, as PCR primers, reverse transcription primers, cloning primers or hybridization probes.

A “polynucleotide sequence” or “nucleotide sequence” or “nucleic acid sequence,” as used interchangeably herein, is the primary sequence of nucleotide residues in a polynucleotide, including of an oligonucleotide, a DNA, and RNA, a nucleic acid, or a character string representing the primary sequence of nucleotide residues, depending on context. From any specified polynucleotide sequence, either the given nucleic acid or the complementary polynucleotide sequence can be determined. Included are DNA or RNA of genomic or synthetic origin which may be single- or double-stranded, and represent the sense or antisense strand. Unless specified otherwise, the left-hand end of any single-stranded polynucleotide sequence discussed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream” sequences; sequence regions on the DNA strand having the same sequence as the RNA transcript that are 3′ to the 3′ end of the RNA transcript are referred to as “downstream” sequences.

“Orientation” refers to the order of nucleotides in a given DNA sequence. For example, an orientation of a DNA sequence in opposite direction in relation to another DNA sequence is one in which the 5′ to 3′ order of the sequence in relation to another sequence is reversed when compared to a point of reference in the DNA from which the sequence was obtained. Such reference points can include the direction of transcription of other specified DNA sequences in the source DNA and/or the origin of replication of replicable vectors containing the sequence. The 5′ to 3′ DNA strand is designated, for a given gene, as “sense,” “plus” or “coding” strand. The complementary 3′ to 5′ strand relative to the “plus” strand is described as “antisense,” “minus” or “not coding.”

As used herein, an “isolated nucleic acid molecule” or “isolated nucleic acid sequence” is a nucleic acid molecule that is either (1) identified and separated from at least one contaminant nucleic acid molecule with which it is ordinarily associated in the natural source of the nucleic acid or (2) cloned, amplified, tagged, or otherwise distinguished from background nucleic acids such that the sequence of the nucleic acid of interest can be determined. An isolated nucleic acid molecule is other than in the form or setting in which it is found in nature. However, an isolated nucleic acid molecule includes a nucleic acid molecule contained in cells that ordinarily express a polypeptide (e.g., an oligopeptide or antibody) where, for example, the nucleic acid molecule is in a chromosomal location different from that of natural cells.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of ribonucleotides along the mRNA chain, and also determines the order of amino acids along the polypeptide (protein) chain. The DNA sequence thus codes for the RNA sequence and for the amino acid sequence.

The term “gene” is used broadly to refer to any nucleic acid associated with a biological function. Genes typically include coding sequences and/or the regulatory sequences required for expression of such coding sequences. The term “gene” applies to a specific genomic or recombinant sequence, as well as to a cDNA or mRNA encoded by that sequence. A “fusion gene” contains a coding region that encodes a polypeptide with portions from different proteins that are not naturally found together, or not found naturally together in the same sequence as present in the encoded fusion protein (i.e., a chimeric protein). Genes also include non-expressed nucleic acid segments that, for example, form recognition sequences for other proteins. Non-expressed regulatory sequences including transcriptional control elements to which regulatory proteins, such as transcription factors, bind, resulting in transcription of adjacent or nearby sequences.

“Expression of a gene” or “expression of a nucleic acid” means transcription of DNA into RNA (optionally including modification of the RNA, e.g., splicing), translation of RNA into a polypeptide (possibly including subsequent post-translational modification of the polypeptide), or both transcription and translation, as indicated by the context.

As used herein the term “coding region” or “coding sequence” when used in reference to a structural gene refers to the nucleotide sequences which encode the amino acids found in the nascent polypeptide as a result of translation of an mRNA molecule. The coding region is bounded, in eukaryotes, on the 5′ side by the nucleotide triplet “ATG” which encodes the initiator methionine and on the 3′ side by one of the three triplets which specify stop codons (i.e., TAA, TAG, TGA).

The term “control sequence” or “control signal” refers to a polynucleotide sequence that can, in a particular host cell, affect the expression and processing of coding sequences to which it is ligated. The nature of such control sequences may depend upon the host organism. In particular embodiments, control sequences for prokaryotes may include a promoter, a ribosomal binding site, and a transcription termination sequence. Control sequences for eukaryotes may include promoters comprising one or a plurality of recognition sites for transcription factors, transcription enhancer sequences or elements, polyadenylation sites, and transcription termination sequences. Control sequences can include leader sequences and/or fusion partner sequences. Promoters and enhancers consist of short arrays of DNA that interact specifically with cellular proteins involved in transcription (Maniatis, et al., Science 236:1237 (1987)). Promoter and regulatory elements have been isolated from a variety of eukaryotic sources including genes in yeast, insect and mammalian cells and viruses (analogous control elements, i.e., promoters, are also found in prokaryotes). The selection of a particular promoter and enhancer depends on what cell type is to be used to express the protein of interest. Some eukaryotic promoters and enhancers have a broad host range while others are functional in a limited subset of cell types (See, Voss, et al., Trends Biochem. Sci., 11:287 (1986) and Maniatis, et al., Science 236:1237 (1987); Magnusson et al., Sustained, high transgene expression in liver with plasmid vectors using optimized promoter-enhancer combinations, Journal of Gene Medicine 13(7-8):382-391 (2011); Xu et al., Optimization of transcriptional regulatory elements for constructing plasmid vectors, Gene. 272(1-2):149-156 (2001)). Enhancers are generally cis-acting, and in nature, are located up to 1 million base pairs away from the expressed gene on a chromosome. In some cases, an enhancer's orientation may be reversed without affecting its function.

The term “vector” means any molecule or entity (e.g., nucleic acid, plasmid, bacteriophage or virus) used to transfer protein coding information into a host cell.

The term “expression vector” or “expression construct” as used herein refers to a recombinant DNA molecule containing a desired coding sequence and appropriate nucleic acid control sequences necessary for the expression of the operably linked coding sequence in a particular host cell. An expression vector can include, but is not limited to, sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired. Such techniques are well known in the art. (E.g., Goodey, Andrew R.; et al., Peptide and DNA sequences, U.S. Pat. No. 5,302,697; Weiner et al., Compositions and methods for protein secretion, U.S. Pat. Nos. 6,022,952 and 6,335,178; Uemura et al., Protein expression vector and utilization thereof, U.S. Pat. No. 7,029,909; Ruben et al., 27 human secreted proteins, US 2003/0104400 A1).

An expression vector contains one or more expression cassettes. An “expression cassette,” at a minimum, contains a promoter, an exogenous gene of interest (“GOI”) to be expressed, and a polyadenylation site and/or other suitable terminator sequence. The promoter typically includes a suitable TATA box or G-C-rich region 5′ to, but not necessarily directly adjacent to, the transcription start site.

The terms “in operable combination”, “in operable order” and “operably linked” as used interchangeably herein refer to the linkage of two or more nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced. For example, a control sequence in a vector that is “operably linked” to a protein coding sequence is ligated thereto so that expression of the protein coding sequence is achieved under conditions compatible with the transcriptional activity of the control sequences. For example, a promoter and/or enhancer sequence, including any combination of cis-acting transcriptional control elements is operably linked to a coding sequence if it stimulates or modulates the transcription of the coding sequence in an appropriate host cell or other expression system. Promoter regulatory sequences that are operably linked to the transcribed gene sequence are physically contiguous to the transcribed sequence, but cis-acting regulatory element sequences that are operably linked to the promoter and/or to a transcribed gene sequence can be operably linked thereto even if the regulatory element is non-contiguous to the promoter sequence and/or transcribed gene sequence. In some useful embodiments of the invention the regulatory element can be situated 5′ to the GAPDH promoter-driven expression cassette, and in other useful embodiments the enhancer can be positioned 3′ to the GAPDH promoter-driven expression cassette.

The term “host cell” means a cell that has been transformed, or is capable of being transformed, with a nucleic acid and thereby expresses a gene of interest. The term includes the progeny of the parent cell, whether or not the progeny is identical in morphology or in genetic make-up to the original parent cell, so long as the gene of interest is present. Any of a large number of available and well-known host cells may be used in the practice of this invention, but a CHO cell line is preferred. The selection of a particular host is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety and costs. A balance of these factors must be struck with the understanding that not all hosts may be equally effective for the expression of a particular DNA sequence. Within these general guidelines, useful microbial host cells in culture include bacteria (such as Escherichia coli sp.), yeast (such as Saccharomyces sp.) and other fungal cells, insect cells, plant cells, mammalian (including human) host cells, e.g., CHO cells and HEK-293 cells. Modifications can be made at the DNA level, as well. The peptide-encoding DNA sequence may be changed to codons more compatible with the chosen host cell. For E. coli, optimized codons are known in the art. Codons can be substituted to eliminate restriction sites or to include silent restriction sites, which may aid in processing of the DNA in the selected host cell. Next, the transformed host is cultured and purified. Host cells may be cultured under conventional fermentation conditions so that the desired compounds are expressed. Such fermentation conditions are well known in the art.

The term “transfection” means the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al., 1981, Gene 13:197. Such techniques can be used to introduce one or more exogenous DNA moieties into suitable host cells.

The term “transformation” refers to a change in a cell's genetic characteristics, and a cell has been transformed when it has been modified to contain new DNA or RNA. For example, a cell is transformed where it is genetically modified from its native state by introducing new genetic material via transfection, transduction, or other techniques. Following transfection or transduction, the transforming DNA may recombine with that of the cell by physically integrating into a chromosome of the cell, or may be maintained transiently as an episomal element without being replicated, or may replicate independently as a plasmid. A cell is considered to have been “stably transformed” when the transforming DNA is replicated with the division of the cell.

A “domain” or “region” (used interchangeably herein) of a protein is any portion of the entire protein, up to and including the complete protein, but typically comprising less than the complete protein. A domain can, but need not, fold independently of the rest of the protein chain and/or be correlated with a particular biological, biochemical, or structural function or location (e.g., a ligand binding domain, or a cytosolic, transmembrane or extracellular domain).

### Selectable Marker(s) Element

Selectable marker genes encode polypeptides necessary for the survival and growth of transfected cells grown in a selective culture medium. Typical selection marker genes encode proteins that (a) confer resistance to antibiotics or other toxins, e.g., ampicillin, tetracycline, or kanomycin for prokaryotic host cells, and neomycin, hygromycin, or methotrexate for mammalian cells; (b) complement auxotrophic deficiencies of the cell; or (c) supply critical nutrients not available from complex media, e.g., the gene encoding D-alanine racemase for cultures of Bacilli.

All of the elements set forth above, as well as others useful in this invention, are well known to the skilled artisan and are described, for example, in Sambrook et al. (Molecular Cloning: A Laboratory Manual, Cold Spring Harbor Laboratory Press, Cold Spring Harbor, N.Y. [1989]) and Berger et al., eds. (Guide to Molecular Cloning Techniques, Academic Press, Inc., San Diego, Calif. [1987]).

### Construction of Cloning Vectors

The cloning vectors most useful for amplification of gene cassettes useful in preparing the recombinant expression constructs of this invention are those that are compatible with prokaryotic cell hosts. However, eukaryotic cell hosts, and vectors compatible with these cells, are within the scope of the invention.

In certain cases, some of the various elements to be contained on the cloning vector may be already present in commercially available cloning or amplification vectors such as pUC18, pUC19, pBR322, the pGEM vectors (Promega Corp, Madison, Wis.), the pBluescript™. vectors such as pBIISK+/− (Stratagene Corp., La Jolla, Calif.), and the like, all of which are suitable for prokaryotic cell hosts. In this case it is necessary to only insert the gene(s) of interest into the vector.

However, where one or more of the elements to be used are not already present on the cloning or amplification vector, they may be individually obtained and ligated into the vector. Methods used for obtaining each of the elements and ligating them are well known to the skilled artisan and are comparable to the methods set forth above for obtaining a gene of interest (i.e., synthesis of the DNA, library screening, and the like).

Vectors used for cloning or amplification of the nucleotide sequences of the gene(s) of interest and/or for transfection of the mammalian host cells are constructed using methods well known in the art. Such methods include, for example, the standard techniques of restriction endonuclease digestion, ligation, agarose and acrylamide gel purification of DNA and/or RNA, column chromatography purification of DNA and/or RNA, phenol/chloroform extraction of DNA, DNA sequencing, polymerase chain reaction amplification, and the like, as set forth in Sambrook et al., supra.

The final vector used to practice this invention is typically constructed from a starting cloning or amplification vector such as a commercially available vector. This vector may or may not contain some of the elements to be included in the completed vector. If none of the desired elements are present in the starting vector, each element may be individually ligated into the vector by cutting the vector with the appropriate restriction endonuclease(s) such that the ends of the element to be ligated in and the ends of the vector are compatible for ligation. In some cases, it may be necessary to “blunt” the ends to be ligated together in order to obtain a satisfactory ligation. Blunting is accomplished by first filling in “sticky ends” using Klenow DNA polymerase or T4 DNA polymerase in the presence of all four nucleotides. This procedure is well known in the art and is described for example in Sambrook et al., supra.

Alternatively, two or more of the elements to be inserted into the vector may first be ligated together (if they are to be positioned adjacent to each other) and then ligated into the vector.

One other method for constructing the vector is to conduct all ligations of the various elements simultaneously in one reaction mixture. Here, many nonsense or nonfunctional vectors will be generated due to improper ligation or insertion of the elements, however the functional vector may be identified and selected by restriction endonuclease digestion.

After the vector has been constructed, it may be transfected into a prokaryotic host cell for amplification. Cells typically used for amplification are E coli DH5-alpha (Gibco/BRL, Grand Island, N.Y.) and other E. coli strains with characteristics similar to DH5-alpha.

Where mammalian host cells are used, cell lines such as Chinese hamster ovary (CHO cells; Urlab et al., Proc. Natl. Acad. Sci USA, 77:4216 [1980])) and human embryonic kidney cell line 293 (Graham et al., J. Gen. Virol., 36:59 [1977]), as well as other lines, are suitable.

Transfection of the vector into the selected host cell line for amplification is accomplished using such methods as calcium phosphate, electroporation, microinjection, lipofection or DEAE-dextran. The method selected will in part be a function of the type of host cell to be transfected. These methods and other suitable methods are well known to the skilled artisan, and are set forth in Sambrook et al., supra.

After culturing the cells long enough for the vector to be sufficiently amplified (usually overnight for E. coli cells), the vector (often termed plasmid at this stage) is isolated from the cells and purified. Typically, the cells are lysed and the plasmid is extracted from other cell contents. Methods suitable for plasmid purification include inter alia the alkaline lysis mini-prep method (Sambrook et al., supra).

### Recombinant Production of Antibodies and Other Polypeptides

Relevant amino acid sequences from an immunoglobulin or polypeptide of interest may be determined by direct protein sequencing, and suitable encoding nucleotide sequences can be designed according to a universal codon table. Alternatively, genomic or cDNA encoding the monoclonal antibodies may be isolated and sequenced from cells producing such antibodies using conventional procedures (e.g., by using oligonucleotide probes that are capable of binding specifically to genes encoding the heavy and light chains of the monoclonal antibodies). Relevant DNA sequences can be determined by direct nucleic acid sequencing.

Cloning of DNA is carried out using standard techniques (see, e.g., Sambrook et al. (1989) Molecular Cloning: A Laboratory Guide, Vols 1-3, Cold Spring Harbor Press, which is incorporated herein by reference). For example, a cDNA library may be constructed by reverse transcription of polyA+ mRNA, preferably membrane-associated mRNA, and the library screened using probes specific for human immunoglobulin polypeptide gene sequences. In one embodiment, however, the polymerase chain reaction (PCR) is used to amplify cDNAs (or portions of full-length cDNAs) encoding an immunoglobulin gene segment of interest (e.g., a light or heavy chain variable segment). The amplified sequences can be readily cloned into any suitable vector, e.g., expression vectors, minigene vectors, or phage display vectors. It will be appreciated that the particular method of cloning used is not critical, so long as it is possible to determine the sequence of some portion of the immunoglobulin polypeptide of interest.

One source for antibody nucleic acids is a hybridoma produced by obtaining a B cell from an animal immunized with the antigen of interest and fusing it to an immortal cell. Alternatively, nucleic acid can be isolated from B cells (or whole spleen) of the immunized animal. Yet another source of nucleic acids encoding antibodies is a library of such nucleic acids generated, for example, through phage display technology. Polynucleotides encoding peptides of interest, e.g., variable region peptides with desired binding characteristics, can be identified by standard techniques such as panning.

The sequence encoding an entire variable region of the immunoglobulin polypeptide may be determined; however, it will sometimes be adequate to sequence only a portion of a variable region, for example, the CDR-encoding portion. Sequencing is carried out using standard techniques (see, e.g., Sambrook et al. (1989) Molecular Cloning: A Laboratory Guide, Vols 1-3, Cold Spring Harbor Press, and Sanger, F. et al. (1977) Proc. Natl. Acad. Sci. USA 74: 5463-5467, which is incorporated herein by reference). By comparing the sequence of the cloned nucleic acid with published sequences of human immunoglobulin genes and cDNAs, one of skill will readily be able to determine, depending on the region sequenced, (i) the germline segment usage of the hybridoma immunoglobulin polypeptide (including the isotype of the heavy chain) and (ii) the sequence of the heavy and light chain variable regions, including sequences resulting from N-region addition and the process of somatic mutation. One source of immunoglobulin gene sequence information is the National Center for Biotechnology Information, National Library of Medicine, National Institutes of Health, Bethesda, Md.

Isolated DNA can be operably linked to control sequences or placed into expression vectors, which are then transfected into host cells that do not otherwise produce immunoglobulin protein, to direct the synthesis of monoclonal antibodies in the recombinant host cells. Recombinant production of antibodies is well known in the art.

Nucleic acid is operably linked when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, operably linked means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

Many vectors are known in the art. Vector components may include one or more of the following: a signal sequence, an origin of replication, one or more selective marker genes (that may, for example, confer antibiotic or other drug resistance, complement auxotrophic deficiencies, or supply critical nutrients not available in the media), an regulatory element, a promoter, and a transcription termination sequence, all of which are well known in the art.

Cell, cell line, and cell culture are often used interchangeably and all such designations herein include progeny. Transformants and transformed cells include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included.

Exemplary host cells include prokaryote, yeast, or higher eukaryote cells. Prokaryotic host cells include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacillus such as B. subtilis and B. licheniformis, Pseudomonas, and Streptomyces. Eukaryotic microbes such as filamentous fungi or yeast are suitable cloning or expression hosts for recombinant polypeptides or antibodies. Saccharomyces cerevisiae, or common baker's yeast, is the most commonly used among lower eukaryotic host microorganisms. However, a number of other genera, species, and strains are commonly available and useful herein, such as Pichia, e.g. P. pastoris, Schizosaccharomyces pombe; Kluyveromyces, Yarrowia; Candida; Trichoderma reesia; Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Host cells for the expression of glycosylated antibodies can be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection of such cells are publicly available, e.g., the L-1 variant of Autographa californica NPV and the Bm-5 strain of Bombyx mori NPV.

Vertebrate host cells are also suitable hosts, and recombinant production of polypeptides (including antibody) from such cells has become routine procedure. Examples of useful mammalian host cell lines are Chinese hamster ovary (CHO) cells of any strain, including but not limited to CHO-K1 cells (ATCC CCL61), DXB-11, CHO-DG-44, CHO-S, CHO-AM1, CHO-DXB11, and Chinese hamster ovary cells/-DHFR (CHO, Urlaub et al., Proc. Natl. Acad. Sci. USA 77: 4216 (1980)); monkey kidney CV1 line transformed by SV40 (COS-7, ATCC CRL 1651); human embryonic kidney line (293 or 293 cells subcloned for growth in suspension culture, [Graham et al., J. Gen Virol. 36: 59 (1977)]; baby hamster kidney cells (BHK, ATCC CCL 10); mouse sertoli cells (TM4, Mather, Biol. Reprod. 23: 243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TRI cells (Mather et al., Annals N.Y Acad. Sci. 383: 44-68 (1982)); MRC 5 cells or FS4 cells; or mammalian myeloma cells.

Host cells are transformed or transfected with the above-described nucleic acids or vectors for production of polypeptides (including antibodies) and are cultured in conventional nutrient media modified as appropriate for inducing promoters, selecting transformants, or amplifying the genes encoding the desired sequences. In addition, novel vectors and transfected cell lines with multiple copies of transcription units separated by a selective marker are particularly useful for the expression of polypeptides, such as antibodies.

The host cells used to produce the polypeptides useful in the invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58: 44 (1979), Barnes et al., Anal. Biochem. 102: 255 (1980), U.S. Pat. Nos. 4,767,704; 4,657,866; 4,927,762; 4,560,655; or 5,122,469; WO90103430; WO 87/00195; or U.S. Patent Re. No. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Upon culturing the host cells, the recombinant polypeptide can be produced intracellularly, in the periplasmic space, or directly secreted into the medium. If the polypeptide, such as an antibody, is produced intracellularly, as a first step, the particulate debris, either host cells or lysed fragments, is removed, for example, by centrifugation or ultrafiltration.

An antibody or antibody fragment) can be purified using, for example, hydroxylapatite chromatography, cation or anion exchange chromatography, or preferably affinity chromatography, using the antigen of interest or protein A or protein G as an affinity ligand. Protein A can be used to purify proteins that include polypeptides are based on human γ1, γ2, or γ4 heavy chains (Lindmark et al., J. Immunol. Meth. 62: 1-13 (1983)). Protein G is recommended for all mouse isotypes and for human γ3 (Guss et al., EMBO J. 5: 15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the protein comprises a C_{H} 3 domain, the Bakerbond ABX™resin (J. T. Baker, Phillipsburg, N.J.) is useful for purification. Other techniques for protein purification such as ethanol precipitation, Reverse Phase HPLC, chromatofocusing, SDS-PAGE, and ammonium sulfate precipitation are also possible depending on the antibody to be recovered.

### Antibody Production by Phage Display Techniques

The development of technologies for making repertoires of recombinant human antibody genes, and the display of the encoded antibody fragments on the surface of filamentous bacteriophage, has provided another means for generating human-derived antibodies. Phage display is described in e.g., Dower et al., WO 91/17271, McCafferty et al., WO 92/01047, and Caton and Koprowski, Proc. Natl. Acad. Sci. USA, 87:6450-6454 (1990), each of which is incorporated herein by reference in its entirety. The antibodies produced by phage technology are usually produced as antigen binding fragments, e.g. Fv or Fab fragments, in bacteria and thus lack effector functions. Effector functions can be introduced by one of two strategies: The fragments can be engineered either into complete antibodies for expression in mammalian cells, or into bispecific antibody fragments with a second binding site capable of triggering an effector function.

Typically, the Fd fragment (V_{H}—C_{H}1) and light chain (V_{L}—C_{L}) of antibodies are separately cloned by PCR and recombined randomly in combinatorial phage display libraries, which can then be selected for binding to a particular antigen. The antibody fragments are expressed on the phage surface, and selection of Fv or Fab (and therefore the phage containing the DNA encoding the antibody fragment) by antigen binding is accomplished through several rounds of antigen binding and re-amplification, a procedure termed panning. Antibody fragments specific for the antigen are enriched and finally isolated.

Phage display techniques can also be used in an approach for the humanization of rodent monoclonal antibodies, called “guided selection” (see Jespers, L. S., et al., Bio/Technology 12, 899-903 (1994)). For this, the Fd fragment of the mouse monoclonal antibody can be displayed in combination with a human light chain library, and the resulting hybrid Fab library may then be selected with antigen. The mouse Fd fragment thereby provides a template to guide the selection. Subsequently, the selected human light chains are combined with a human Fd fragment library. Selection of the resulting library yields entirely human Fab.

A variety of procedures have been described for deriving human antibodies from phage-display libraries (See, for example, Hoogenboom et al., J. Mol. Biol., 227:381 (1991); Marks et al., J. Mol. Biol, 222:581-597 (1991); U.S. Pat. Nos. 5,565,332 and 5,573,905; Clackson, T., and Wells, J. A., TIBTECH 12, 173-184 (1994)). In particular, in vitro selection and evolution of antibodies derived from phage display libraries has become a powerful tool (See Burton, D. R., and Barbas Ill, C. F., Adv. Immunol. 57, 191-280 (1994); and, Winter, G., et al., Annu. Rev. Immunol. 12, 433-455 (1994); U.S. patent application no. 20020004215 and WO92/01047; U.S. patent application no. 20030190317 published Oct. 9, 2003 and U.S. Pat. Nos. 6,054,287; 5,877,293. Watkins, “Screening of Phage-Expressed Antibody Libraries by Capture Lift,” Methods in Molecular Biology, Antibody Phage Display: Methods and Protocols 178: 187-193, and U.S. Patent Application Publication No. 20030044772 published Mar. 6, 2003 describes methods for screening phage-expressed antibody libraries or other binding molecules by capture lift, a method involving immobilization of the candidate binding molecules on a solid support.

### Fluidic Devices

Fluidic devices refer to an apparatus that use small amounts of fluid to carry out various types of analysis. The fluidic device comprises one or more discrete circuits configured to hold a fluid, each circuit comprised of fluidically interconnected circuit elements. The circuit element including but not limited to region(s), flow path(s), channel(s), chamber(s), and/or pen(s), and at least one port configured to allow the fluid to flow into and/or out of the fluidic device. These devices use chips, cells, channel, or sequestrationpens that contain the fluid for analysis.

Fluidic devices such as microfluidic devices generally have one or more channels with at least one dimension less than 1 mm. Common fluids used in fluidic devices include whole blood samples, bacterial cell suspensions, protein or antibody solutions and various buffers. Fluidic devices can be used to obtain a variety of measurements including molecular diffusion coefficients, fluid viscosity, pH, chemical binding coefficients and enzyme reaction kinetics. Other applications for fluidic devices include capillary electrophoresis, isoelectric focusing, immunoassays, flow cytometry, sample injection of proteins for analysis via mass spectrometry, PCR amplification, DNA analysis, cell manipulation, cell separation, cell patterning and chemical gradient formation. Many of these applications have utility for clinical diagnostics.

The advantages for using fluidic devices include that the volume of fluids within these channels is very small, usually several nanoliters, and the amounts of reagents and analytes used is quite small. Moreover, when analyzing protein-producing cells, a relatively small number of cells (or even single cells) can produce a sufficient quantity and concentration of protein for analysis, reducing or avoiding incubation times for colony expansion. The fabrication techniques used to construct microfluidic devices are relatively inexpensive and are very amenable both to highly elaborate, multiplexed devices and also to mass production. Fluidic technologies enable the fabrication of highly integrated devices for performing several different functions on the same support chip.

Any fluidic device can be used (or modified to be used) in the disclosed methods, including commercially available devices. The fluidic device may be configured for use in an optofluidic system, which can use light to manipulate matter in the fluidic device such as cells.

### EXAMPLES

### Example 1

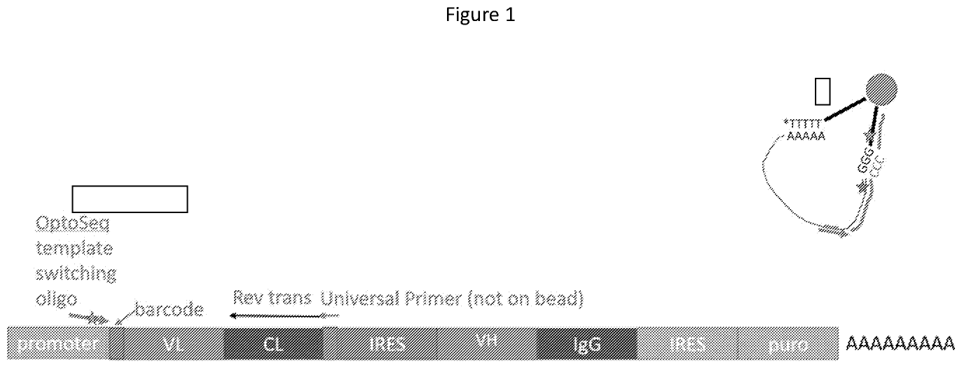

An exemplary polynucleotide molecule expressing a monoclonal antibody of the disclosure is provided in the schematic of FIG. 1. The polynucleotide molecule is designed to express a monoclonal antibody and the polynucleotide molecule comprises a polynucleotide sequence encoding the variable domain of the light chain of the monoclonal antibody, a polynucleotide sequence encoding the constant domain of the light chain and a polynucleotide sequence encoding the heavy chain of the monoclonal antibody. This polynucleotide molecule is designed to include an optical barcode and a unique molecular identifier barcode upstream from the polynucleotide sequence encoding the variable domain of the light chain. In the exemplary polynucleotide molecule, the optical barcode (when added) and the unique molecular identifier (UMI) barcode are positioned adjacent to each other and immediately upstream form the polynucleotide sequence encoding the variable domain of the light chain of the monoclonal antibody. The exemplary polynucleotide molecule also comprises two IRES, one that is immediately downstream from the specific sequence for the universal primer.

For the exemplary polynucleotide molecule, the optical barcode is part of a template switching oligonucleotide. This template switching oligonucleotide is conjugated to a dual primer bead which further comprises an oligo dT sequence that will bind to the mRNA. The universal primer will initiate extension closer to the 5′ end. In the example, the positioning of the universal primer downstream of the polynucleotide sequence encoding the constant domain of a light chain of an antibody protein produce limits reverse transcription to a short number of nucleotides (about 700 nucleotides) that comprise the UMI barcode and the optical barcode.

### Example 2

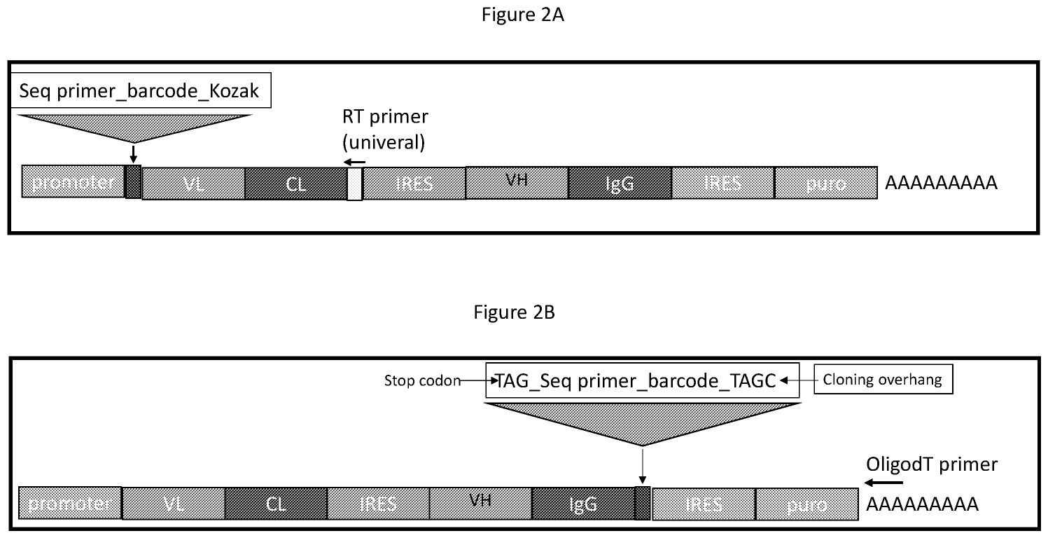

In addition, some polynucleotide molecules disclosed herein are designed for use in on chip sequencing-by-synthesis, see e.g. the schematic of FIG. 2A-B.

In one example, the polynucleotide molecule is designed to express a monoclonal antibody and the polynucleotide molecule comprises a polynucleotide sequence encoding the variable domain of the light chain of the monoclonal antibody, a polynucleotide sequence encoding the constant domain of the light chain and a polynucleotide sequence encoding the heavy chain of the monoclonal antibody. This polynucleotide molecule is designed to include an oligonucleotide sequence specific of a sequencing primer immediately upstream of a unique molecular identifier barcode immediately upstream from the Kozak sequence and the polynucleotide sequence encoding the variable domain of the light chain. The sequencing primer may anneal to the Kozak sequence. In the exemplary polynucleotide molecule, the site for the sequencing primer and the unique molecular identifier barcode are positioned adjacent to each other and immediately upstream from the polynucleotide sequence encoding the variable domain of the light chain of the monoclonal antibody. The exemplary polynucleotide molecule also comprises two IRES, one that is immediately downstream from the specific sequence for the universal RT primer. The universal RT primer is thus disposed to reverse-transcribe a relatively short nucleic acid sequence comprising the UMI barcode, and is suitable for on-chip sequencing. See FIG. 2A.

When sequencing this polynucleotide molecule (See FIG. 2A), the universal oligodT primer will capture the mRNA and permit reverse transcription that includes the UMI barcode. The positioning of the sequencing primer will allow for sequencing up to 15 bases and the reagents will be diffuse in and out of locations on a fluidic device (such as sequestration pens), allowing for on-chip sequencing in the fluidic device. As such, the sequencing primer site adjacent to the UMI barcode permits sequencing in situ (e.g., on-chip, such as in a fluidic device).

This template switching oligo nucleotide is part of a dual primer bead which comprises an oligo dT sequence that will bind to the mRNA. The universal primer will initiate extension closer to the 5′ end. In the example, the positioning of the universal primer downstream of the polynucleotide sequence encoding the constant domain of a light chain of an antibody protein produce limits reverse transcription to a short number of nucleotides to 700 nucleotides.

In an exemplary polynucleotide molecule, a sequencing primer anneals downstream of a stop codon, e.g. TAG, and upstream of the UMI. A cloning overhang is positioned downstream of the sequencing primer annealing site, between the nucleic acid sequence encoding the heavy chain polypeptide and an IRES (see FIG. 2B).

### Example 3

The polynucleotide molecule described in Example 1 are designed for the sequencing reaction in an optical fluidic device, such as in a sequestration pen. Because cDNAs from multiple pens are pooled for export and samples are fragmented during sequencing, under current protocols sequencing is limited to 500nt at 5′ or 3′ ends.

As an alternative, the cDNA is produced and then the cDNA is exported. Long read sequencing is subsequently performed to verify the whole molecule sequence. This method would not rely on barcoding. Long read sequencing may be useful for pooled cloning strategies.

Long read sequencing allows for directly sequence a polynucleotide molecules in real time, without the need for amplification. This direct sequencing approach enables the production of reads that are considerably longer than those resulting from short read sequencing. Alternatively, ‘synthetic’ long-read sequencing approaches utilise modified sample processing and conventional short read sequencing to computationally reconstruct long reads from shorter sequencing reads.

### Example 4

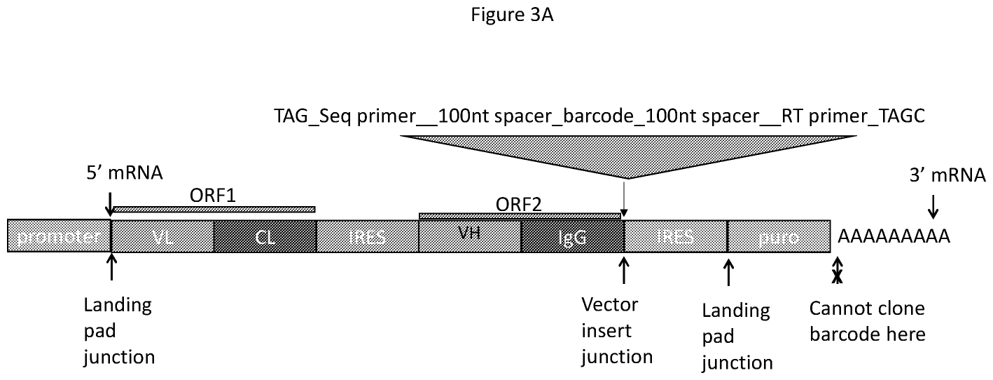

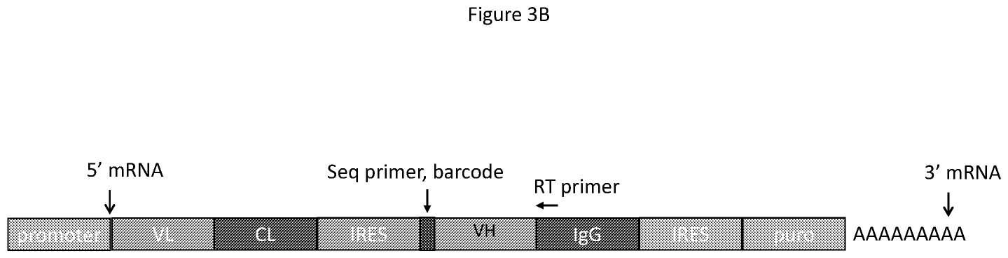

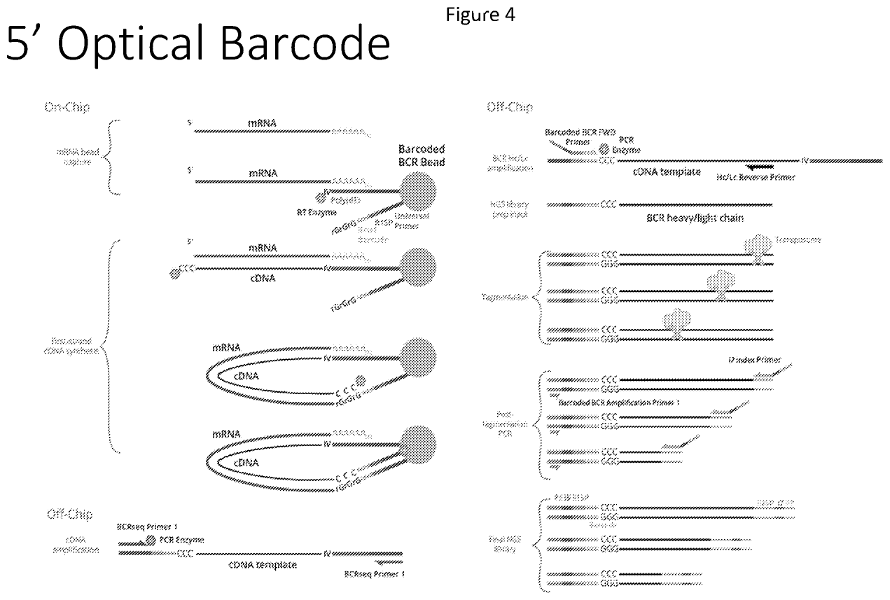

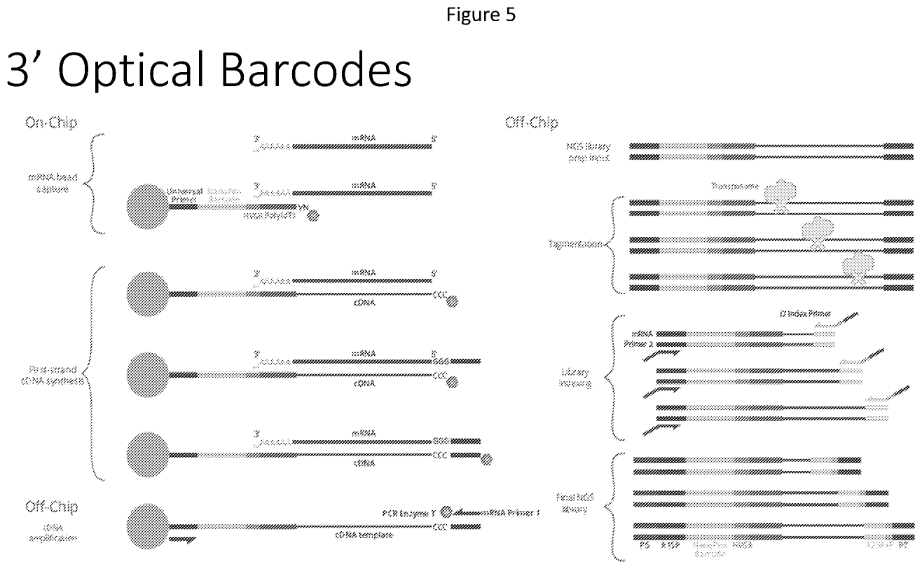

Additional exemplary polynucleotide molecules described herein are designed in view of a landing pad construct that has the polyadenylation sequence in the cell host landing pad, far downstream of the insert junction. As shown in the schematic of FIG. 3A, a barcode is inserted downstream of the polynucleotide encoding the heavy chain polypeptide. At this position, a custom on-bead RT primer & custom sequence primer oligonucleotide is inserted. An exemplary insert comprises a sequencing primer comprising a stop codon (TAG) with a 100 nucleotide spacer, a unique molecular identification (UMI) barcode, 100 nucleotide spacer and a RT optical barcode. Because the optical barcode ends up 500 bases from the UMI barcode, unfragmented amplicon PE sequencing is then carried out using PCR (Tagmentation would be unavailable due to the spacing between the optical barcode and UMI barcode). Additionally, efficiency of custom RT primer is expected to be lower than for oligodT.

Another exemplary polynucleotide molecule is provided in FIG. 3B. In this polynucleotide molecule, a custom oligo is in the sequence encoding the CH1 domain which is upstream of the polynucleotide sequence encoding the light chain polypeptide. The advantage is that the barcode would be adjacent to the polynucleotide sequence encoding the heavy chain polypeptide. However, the disadvantage are this design is that the custom RT primer is on-bead, with optical barcodes. This arrangement is limiting, as the RT primer sequence would have to be chosen from native sequence, which imposes strong constraints upon possible primer sequences, and may lower efficiency. Because the optical barcode ends up 500 bases from the UMI barcode, unfragmented amplicon PE sequencing is then carried out using PCR.

What is claimed is:

1. A polynucleotide molecule encoding an antibody protein product, wherein the polynucleotide molecule comprises i) a nucleotide sequence specific for the addition of a molecular barcode by a template switching reverse transcriptase (RT), ii) a unique molecular identifier (UMI) barcode that is different than the molecular barcode, iii) a nucleotide sequence encoding a light chain polypeptide of the antibody protein product, iv) a nucleotide sequence encoding a heavy chain polypeptide of the antibody protein product, and v) a nucleotide sequence specific for a universal RT primer.

2. The polynucleotide molecule of claim 1, wherein the molecular barcode is an optical barcode.

3. The polynucleotide molecule of claim 2, wherein the optical barcode (i) is identifiable by determining its polynucleotide sequence, and (ii) is identifiable by annealing to a polynucleotide probe comprising one or more optical moieties.

4. The polynucleotide molecule of any of the preceding claims, wherein the molecular barcode or the UMI barcode comprises a random 6mer, 7mer, 8mer, 9mer, 10mer, 11mer, 12mer, 13mer, 14mer or 15mer.

5. The polynucleotide molecule of any of the preceding claims, wherein the polynucleotide molecule further comprises a promoter sequence.

6. The polynucleotide molecule of any of the preceding claims, wherein the polynucleotide molecule further comprises at least two internal ribosome entry site (IRES) sequences; or
wherein the polynucleotide molecule comprises at least one internal ribosome entry site IRES sequence and at least one promoter; or
wherein the polynucleotide molecule comprises at least two promoters.

7. The polynucleotide molecule of any of the preceding claims, further comprising a nucleotide encoding a selection gene product, such as puromycin-N-acetyltransferase.

8. The polynucleotide molecule of any of the preceding claims, wherein the nucleotide sequence specific for the universal RT primer is disposed between the nucleotide sequence encoding the light chain polypeptide and the nucleotide sequence encoding the heavy chain polypeptide.

9. The polynucleotide molecule of any of the preceding claims, wherein the nucleotide sequence specific for the universal RT primer is downstream of the nucleotide sequence encoding the light chain polypeptide of the antibody protein product.

10. The polynucleotide molecule of any of the preceding claims, wherein the nucleotide sequence specific for the addition of a molecular barcode by a template switching reverse transcriptase is adjacent to the UMI barcode.

11. The polynucleotide molecule of any of the preceding claims, wherein the nucleotide sequence specific for the addition of a molecular barcode by a template switching reverse transcriptase is immediately upstream of the UMI barcode.

12. The polynucleotide molecule of any of the preceding claims, wherein the UMI barcode is immediately upstream from the nucleotide sequence encoding the light chain polypeptide of the antibody protein product.

13. The polynucleotide molecule of any of claims 10-12, wherein the molecular barcode is an optical barcode.

14. The polynucleotide molecule of any of the preceding claims wherein the nucleotide sequence specific for the RT universal primer is immediately downstream of the nucleotide sequence encoding the light chain polypeptide of the antibody protein product.

15. The polynucleotide molecule of any of the preceding claims wherein an IRES is immediately downstream of the sequence specific for the RT universal primer.

16. The polynucleotide molecule of any of the preceding claims wherein an IRES is immediately downstream of the nucleotide sequence encoding the heavy chain polypeptide of the antibody protein product.

17. The polynucleotide molecule of any of the preceding claims, wherein the heavy chain polypeptide comprises a heavy chain variable region; and the light chain polypeptide comprises a light chain variable region.

18. The polynucleotide molecule of any of the preceding claims, wherein:
(a) the nucleotide sequence encoding the light chain polypeptide of the antibody protein product encodes: a light chain variable region, and a light chain constant region immediately downstream of the light chain constant region; and
(b) the nucleotide sequence encoding the heavy chain polypeptide of the antibody protein product encodes: a heavy chain variable region, and a heavy chain constant region immediately downstream of the heavy chain constant region.

19. The polynucleotide molecule of any of the preceding claims, wherein the nucleotide sequence encoding the light chain polypeptide of the antibody protein product is upstream of the nucleotide sequence encoding the heavy chain polypeptide of the antibody protein product.

20. The polynucleotide molecule of any of claims 1-18, wherein the nucleotide sequence encoding the light chain polypeptide of the antibody protein product is downstream of the nucleotide sequence encoding the heavy chain polypeptide of the antibody protein product.

21. A polynucleotide molecule encoding an antibody protein product, wherein the polynucleotide molecule comprises in the 5′ to 3′ direction: i) a promoter, ii) a nucleotide sequence specific for the addition of a molecular barcode by a template switching reverse transcriptase, iii) a unique molecular identifier (UMI) barcode, iv) a nucleotide sequence encoding a the first polypeptide of an antibody protein product, v) a nucleotide sequence specific for a reverse transcriptase (RT) universal primer, vi) a first IRES, vii) a nucleotide sequence encoding a second polypeptide of the antibody protein product, viii) a nucleotide sequence encoding the constant domain of the heavy chain of the antibody protein product, ix) a second IRES and x) a nucleotide sequence encoding a selection gene product, such as puromycin-N-acetyltransferase.

22. A polynucleotide molecule encoding an antibody protein product, wherein the polynucleotide sequence comprises, i) a sequencing primer annealing site, ii) a unique molecular identifier barcode, iii) a nucleotide sequence encoding a light chain polypeptide of the antibody protein product, iv) a nucleotide sequence encoding a heavy chain polypeptide of the antibody protein product; and iv) a nucleotide sequence specific for a universal reverse transcriptase (RT) primer.

23. The polynucleotide molecule of claim 22, wherein the polynucleotide molecule further comprises a promoter sequence.

24. The polynucleotide molecule of claim 22 or 23, wherein the polynucleotide molecule further comprises at least two internal ribosome entry site (IRES) sequences

25. The polynucleotide molecule of any one of claims 22-24, further comprising a nucleotide encoding a selection gene product, such as puromycin-N-acetyltransferase.

26. The polynucleotide molecule of any one of claims 22-25, wherein the nucleotide sequence specific for the universal RT primer is disposed between the nucleotide sequence encoding the light chain polypeptide and the nucleotide sequence encoding the heavy chain polypeptide.

27. The polynucleotide molecule of any one of claims 22-26, wherein the nucleotide sequence specific for the universal RT primer is downstream of the nucleotide sequence encoding the light chain polypeptide of the antibody protein product.

28. The polynucleotide molecule of any one of claims 22-27, wherein the nucleotide sequence specific for the RT universal primer is immediately downstream of the nucleotide sequence encoding the light chain polypeptide of the antibody protein product.

29. The polynucleotide molecule of any one of claims 22-28, wherein an IRES is immediately downstream of the sequence specific for the RT universal primer.

30. The polynucleotide molecule of any of claims 22-29, wherein an IRES is immediately downstream of the nucleotide sequence encoding the heavy chain polypeptide of the antibody protein product.

31. The polynucleotide molecule of any of the preceding claims, wherein nucleotide sequence specific for an optical barcode is configured to receive the optical barcode conjugated to a solid support.

32. The polynucleotide molecule of any of the preceding claims wherein the nucleic acid specific for the addition of a molecular barcode by a template switching reverse transcriptase is configured for the addition of the optical barcode from by a template switching oligonucleotide from a template optical barcode conjugated to a solid support.

33. The polynucleotide molecule of any of the preceding claims wherein the 5′ end of the nucleotide sequence specific the for the addition of a molecular barcode comprises the polynucleotide sequence CCC.

34. The polynucleotide molecule of any of the preceding claims wherein the nucleotide barcode is configured for reverse transcription by template switching reverse transcriptase primed by the RT universal primer.

35. The polynucleotide molecule of any of the preceding claims wherein the nucleotide sequence specific for a reverse transcriptase (RT) universal primer is configured to anneal to free universal primer that is not disposed on the solid support, and wherein the annealed universal primer is configured for reverse transcription of the nucleotide barcode by the reverse transcriptase.

36. The polynucleotide molecule of any of claims 31-35, wherein the solid support is a bead, resin or agarose.

37. The polynucleotide molecule of any of claims 22-36, wherein:
(a) the first polypeptide of the antibody protein product comprises a light chain polypeptide and the second polypeptide of the antibody protein product comprises a heavy chain polypeptide; or
(b) the first polypeptide of the antibody protein product comprises a heavy chain polypeptide and the second polypeptide of the antibody protein product comprises a light chain polypeptide.

38. The polynucleotide molecule of claim 37, wherein the light chain polypeptide comprises a light chain variable region, and wherein the heavy chain polypeptide comprises a heavy chain variable region.

39. The polynucleotide molecule of claim 37 or 38, wherein:
the nucleotide sequence encoding the light chain polypeptide of the antibody protein product encodes: a light chain variable region, and a light chain constant region immediately downstream of the light chain constant region; and
the nucleotide sequence encoding the heavy chain polypeptide of the antibody protein product encodes: a heavy chain variable region, and a heavy chain constant region immediately downstream of the heavy chain constant region.

40. The polynucleotide molecule encoding an antibody protein product wherein the antibody protein product comprises or consists of a large peptide, antibody, antibody fragment, antibody fusion peptide or antigen-binding fragment thereof.

41. The polynucleotide molecule of claim 40, wherein the antibody is a polyclonal or monoclonal antibody.

42. A method of screening clones expressing an antibody protein product comprising pooling clones comprising a polynucleotide molecule of any one of the preceding claims, polymerizing a DNA of the pooled clones and sequencing the DNA in a fluidic device.

43. The method of claim 42, wherein polymerizing the DNA of the pooled clones comprises reverse transcribing the polynucleotide molecule with a template switching reverse transcriptase.

44. A method comprising:
annealing the polynucleotide molecule of any one of claims 1-33 to template comprising optical barcode,
annealing a universal reverse transcriptase primer to the polynucleotide molecule;
extending the annealed universal reverse transcriptase primer with a template switching reverse transcriptase, thereby producing a cDNA of the polynucleotide molecule comprising the UMI barcode and the optical barcode.

45. The method of claim 44, wherein the method is performed in a fluidic device.

46. The method of any of claims claim 42-45, further comprising detecting the presence of a molecular barcode and/or a UMI barcode.

47. The method of any of claims 43-46, wherein the fluidic device comprises or consists of a microfluidic chip or sequestration pen.
